(19) United States
(12) Patent Application Publication
(10) Pub. No.: US 2016/0007911 A1
(43) Pub. Date: Jan. 14, 2016

(54) BREATHING GUIDANCE SYSTEM AND METHOD HAVING ACTIVE BIOFEEDBACK MECHANISM

(71) Applicant: HTC Corporation, Taoyuan County (TW)

(72) Inventors: Chun-Yih WU, Taoyuan County (TW); Ta-Chun PU, Taoyuan County (TW); Yen-Liang KUO, Taoyuan County (TW); Chien-Ting HO, Taoyuan County (TW)

(21) Appl. No.: 14/330,003

(22) Filed: Jul. 14, 2014

(51) Int. Cl. A61B 5/00; A61B 5/11; A61B 7/00; A61B 5/05; A61B 8/08; A61B 5/0205; A61B 5/145

(52) U.S. Cl. CPC A61B 5/486; A61B 5/0205; A61B 5/11; A61B 5/14542; A61B 5/0059; A61B 5/05; A61B 8/08; A61B 7/003; A61B 5/7445; A61B 5/7415; A61B 5/0816

(57) ABSTRACT

A breathing guidance system having an active biofeedback mechanism is provided. The breathing guidance system includes a physiological status detection device, a processing device, a breathing indication device and an instant breathing detection module. The physiological status detection device detects at least one of instant physiological status information of the user. The processing device generates an ideal breathing waveform according to the instant physiological status information to further generate a control signal. The breathing indication device generates a breathing indication signal according to the control signal. The instant breathing detection module detects instant breathing status information of the user. The processing device further generates an instant breathing waveform according to the instant breathing status information to compare the ideal breathing waveform and the instant breathing waveform to generate a breathing condition indication signal.

### BACKGROUND

1. Field of Invention

The present invention relates to a breathing guidance technology. More particularly, the present invention relates to a breathing guidance system and method having active biofeedback mechanism.

2. Description of Related Art

In the current industrial and commercial society, long duty hours and high pressures make people hard to relax. Therefore, lots of breathing methods related with Tai-Chi, Kung-Fu or Yoga are used to train people the technique of breathing to relax their mind and body.

However, the current guidance method is a one-way guidance method such that the user only passively receives information. For example, the user may read documents, web pages or directions of application software and follow the guidance according to the easy steps obtained therefrom. Nevertheless, the user does not really know whether the breathing performed according to these steps is correct or not when only the graphs or the texts are provided. If the breathing performed by the user is wrong, not only the user is not able to relax, it may further do harm to the body of the user.

Accordingly, what is needed is a breathing guidance system and method having active biofeedback mechanism to addresses the drawbacks of the passive guidance method.

### SUMMARY

The invention provides a breathing guidance system having an active biofeedback mechanism. The breathing guidance system includes a physiological status detection device, a processing device, a breathing indication device and an instant breathing detection module. The physiological status detection device detects at least one of instant physiological status information of a user. The processing device generates a target breathing waveform according to the instant physiological status information and a default breathing waveform to further generate a control signal. The breathing indication device generates a breathing indication signal according to the control signal. The instant breathing detection module detects instant breathing status information of the user. The processing device further generates an instant breathing waveform according to the instant breathing status information and the default breathing waveform to compare the target breathing waveform and the instant breathing waveform to generate a breathing condition indication signal.

Another aspect of the present invention is to provide a breathing guidance method used in a breathing guidance system having an active biofeedback mechanism. The breathing guidance method includes the steps outlined below. At least one of instant physiological status information of a user is detected by a physiological status detection device. A target breathing waveform is generated according to the instant physiological status information and a default breathing waveform by a processing device to further generate a control signal. A breathing indication signal is generated according to the control signal by a breathing indication device. The instant breathing status information of the user is detected by an instant breathing detection module. An instant breathing waveform is generated according to the instant breathing status information and the default breathing waveform by the processing device to compare the target breathing waveform and the instant breathing waveform to generate a breathing condition indication signal.

These and other features, aspects, and advantages of the present invention will become better understood with reference to the following description and appended claims.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiment, with reference made to the accompanying drawings as follows:

FIG. 1 is a block diagram of a breathing guidance system having an active biofeedback mechanism in an embodiment of the present invention;

FIG. 2 is a block diagram of a breathing guidance system having an active biofeedback mechanism in an embodiment of the present invention; and

FIG. 3 is a flow chart of a breathing guidance method in an embodiment of the present invention.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

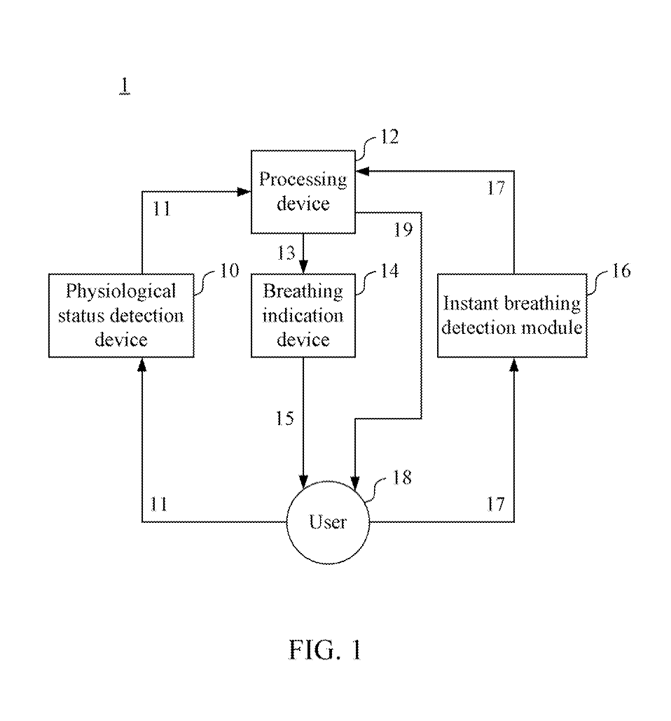

FIG. 1 is a block diagram of a breathing guidance system 1 having an active biofeedback mechanism in an embodiment of the present invention. The breathing guidance system 1 includes a physiological status detection device 10, a processing device 12, a breathing indication device 14 and an instant breathing detection module 16.

The physiological status detection device 10 detects at least one of instant physiological status information 11 of a user 18. In different embodiments, the physiological status detection device 10 can be a specific device to detect a single type of the instant physiological status information 11. For example, the physiological status detection device 10 can be such as, but not limited to a gravity sensor, an oxygen saturation measuring device or a heartbeat rate measuring device.

The gravity sensor can detect the change of the gravity direction to detect body movement information as the instant physiological status information 11. The body movement information may include such as, but not limited to a direction and a distance of the body movement of the user 18.

The oxygen saturation measuring device can detect oxygen saturation information as the instant physiological status information 11. The oxygen saturation is SO2, which is the percentage of the content of oxyhemoglobin (HbO2) that is already combined with oxygen to total hemoglobin (Hb) that can be combined with oxygen. The oxygen saturation is an important biological parameter of the circulation breath.

The heartbeat rate measuring device can detect the pulse or heartbeat rate information as the instant physiological status information 11.

In other embodiments, the physiological status detection device 10 can be other types of detection devices. In some embodiments, the physiological status detection device 10 may include at least two of the devices mentioned above to become a compound detection device to detect a multiple types of the instant physiological status information 11. Moreover, the physiological status detection device 10 can perform the detection by using a contacting way or a non-contacting way according to different types of detection methods to detect the instant physiological status information 11. For example, the physiological status detection device 10 can be a watch-like contacting detection device wore by the user 18, or a seaming non-contacting detection device disposed at a specific distance apart from the user 18.

In an embodiment, the processing device 12 can be a handheld electronic device such as, but not limited to a smartphone or a tablet PC. The processing device 12 can receive the instant physiological status information 11 by using either a wired or a wireless transmission method to generate a target breathing waveform according to the instant physiological status information 11 and a pre-stored default breathing waveform (not illustrated). In an embodiment, the default breathing waveform is stored in such as, but not limited to a storage element (not illustrated) of the processing device 12 such that the default breathing waveform is retrieved by the processing device 12. In an embodiment, the default breathing waveform corresponds to different subjects such as sleeping, sporting or exercising. In an embodiment, the processing device 12 provides options to the user to select and receives the selection command to select one of a plurality of subjects to generate the default breathing waveform.

In an embodiment, the processing device 12 performs evaluation according to the instant physiological status information 11 to generate the weighting values of different instant physiological status information 11. In an embodiment, the processing device 12 performs a comparison based on self-correlation and cross-correlation on different instant physiological status information 11 to generate time-domain related coefficients and further perform conversion to generate frequency-domain related coefficients. The weighting values are assigned according to the time-domain related coefficients and the frequency-domain related coefficients. In an embodiment, the processing device 12 further adjusts the default breathing waveform to generate the target breathing waveform according to the weighted time-domain related coefficients and the frequency-domain related coefficients. In an embodiment, the target breathing waveform includes a breathing depth, a breathing frequency or a combination of the above.

The processing device 12 further performs calculation based on the pre-stored algorithm according to the instant physiological status information 11 and the weighting values to generate the target breathing waveform that is suitable for the user 18. Different pre-stored algorithms correspond to different breathing methods such as, but not limited to Tai-Chi, Kung-Fu or Yoga. The target breathing waveform includes a breathing depth, a breathing frequency or a combination of the above. Moreover, the processing device 12 generates a control signal 13 according to the target breathing waveform.

The breathing indication device 14 generates a breathing indication signal 15 according to the control signal 13. In an embodiment, the breathing indication device 14 is a light generation device such as, but not limited to a light emitting diode (LED) or other illumination elements.

The light generation device can generates a breathing indication light having an indication color temperature, an indication flashing frequency or a combination of the above according to the control signal, wherein the indication color temperature and/or the indication flashing frequency is related to the target breathing waveform. For example, the indication color temperature can be related to the depth of the breathing. The higher color temperature indicates that the depth of the breathing needs to be adjusted to be deeper and the lower color temperature indicates that the depth of the breathing needs to be adjusted to be shallower. The indication flashing frequency can be related to the frequency of the breathing. The higher flashing frequency indicates that the frequency of the breathing needs to be adjusted to be higher and the lower flashing frequency indicates that the frequency of the breathing needs to be adjusted to be lower.

It is noted that the method of relating the color temperature and the flashing frequency to the depth and the frequency of the breathing respectively is merely an example. In other embodiments, other parameters of the light generation device can be used to correspond to the breathing parameters.

In another embodiment, the breathing indication device 14 can be a sound generation device such as, but not limited to a speaker, an amplifier or other sound-generating elements. The sound generation device generates a single frequency indication sound signal or a music indication sound signal that includes an indication intensity, an indication frequency or a combination of the same according to the control signal 13, wherein the indication intensity and/or the indication frequency is related to the target breathing waveform.

For example, the indication intensity can be related to the depth of the breathing. The larger indication intensity (such as volume) indicates that the depth of the breathing needs to be adjusted to be deeper and the smaller indication intensity indicates that the depth of the breathing needs to be adjusted to be shallower. The indication frequency can be related to the frequency of the breathing. The higher indication frequency indicates that the frequency of the breathing needs to be adjusted to be higher and the lower indication frequency indicates that the frequency of the breathing needs to be adjusted to be lower. The single frequency indication sound signal can perform the indication of the breathing of the user by using merely the intensity and the frequency mentioned above. On the other hand, the music indication sound signal can perform the indication of the breathing of the user by using the music including various combinations of speed, intensity and melody.

It is noted that the method of relating the indication intensity and the indication frequency to the depth and the frequency of the breathing respectively is merely an example. In other embodiments, other parameters of the sound generation device can be used to correspond to the breathing parameters.

The user 18 can gradually adjust the frequency or the depth of the breathing by using the breathing indication signal 15 in the form of light or sound to further match the breathing activity corresponding to the target breathing waveform mentioned above.

The instant breathing detection module 16 detects instant breathing status information 17 of the user 18. In an embodiment, the instant breathing status information 17 includes a breathing depth, a breathing frequency or a combination of the above.

In an embodiment, the instant breathing detection module 16 includes such as, but not limited to a radar wave transceiver module, a supersonic wave transceiver module or a laser transceiver module to transmit a radar wave, a supersonic wave or a laser to the user or receive the radar wave, the supersonic wave or the laser from the user to detect the instant breathing status information 17 of the user 18. For example, the radar wave transceiver module may transmit a radar wave having a frequency of 30 MHz to the chest, the stomach or other body portions that displace according to the breathing to receive the radar wave bounced back. The time difference of the reception of the radar wave is used to determine the distance of expansion and contraction of the chest and the stomach.

In another embodiment, the instant breathing detection module 16 includes a sound-receiving module to receive a breathing sound of the user 18 to determine the frequency and the strength of the breathing sound to further determine the depth and the frequency of the breathing.

The processing device 12 further generates an instant breathing waveform according to the instant breathing status information 17 and the default breathing waveform to compare the target breathing waveform and the instant breathing waveform generated according to the instant breathing status information 17 to further generate a breathing condition indication signal 19. In an embodiment, similar to the target breathing waveform, the instant breathing waveform includes a breathing depth, a breathing frequency or a combination of the same.

In an embodiment, the breathing condition indication signal 19 can be displayed by such as, but not limited to a display module (not illustrated) disposed in the processing device 12. An interactive content of a graphic form or a text for is used to indicate the user how to perform the adjustment on the breathing. In other embodiments, the breathing condition indication signal 19 can be generated as an indication sound or an indication light by using the speaker or the light-emitting element (not illustrated) disposed in the processing module 12 to indicate the user how to keep performing the adjustment on the breathing or indicate the difference between the target breathing condition and the current breathing condition.

It is noted that in an embodiment, the processing device 12 can determine that whether a breathing waveform difference between the instant breathing waveform and the default breathing waveform is smaller than a threshold value in advance. The breathing condition of the user matches the ideal breathing condition when the breathing waveform difference is smaller than the threshold value. As a result, the processing device 12 does not need to generate the target breathing waveform. The breathing condition of the user does not match the ideal breathing condition when the breathing waveform difference is not smaller than the threshold value. The processing device 12 further generates the target breathing waveform according to the breathing waveform difference.

Consequently, the advantage of the breathing guidance system 1 of the present invention makes use of the active biofeedback mechanism to obtain the instant physiological status information and the breathing condition to perform comparison and further provide stimulus by using the indication signal in the form of such as, but not limited to sound or light to guide the user to become stable.

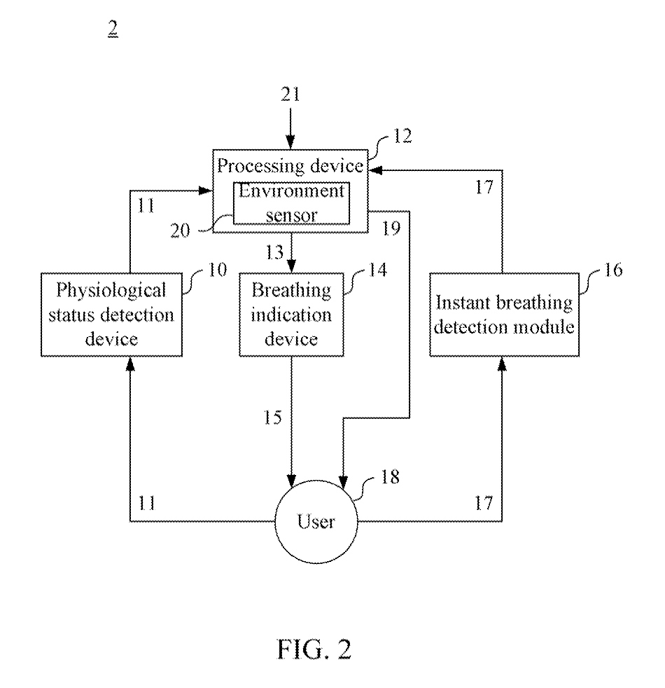

FIG. 2 is a block diagram of a breathing guidance system 2 having an active biofeedback mechanism in an embodiment of the present invention. The breathing guidance system 2 substantially includes the same components as the breathing guidance system 1 illustrated in FIG. 1, such as the physiological status detection device 10, the processing device 12, the breathing indication device 14 and the instant breathing detection module 16. As a result, the detail of the identical components is not further described herein.

In the present embodiment, the processing device 12 of the breathing guidance system 2 further includes an environment sensor 20. The environment sensor 20 senses actual environment status information 21 of an environment where the processing device 12 is located. The processing device 12 generates the default breathing waveform according to the actual environment status information 21 and the instant physiological status information 11. The actual environment status information 21 includes light intensity, temperature, humidity, carbon monoxide content, carbon dioxide content, volatile organic compound content, formaldehyde content or a combination of the above.

Furthermore, after retrieving the actual environment status information 21, the processing device 12 can selectively generates an indication message (not illustrated) such as, but not limited to the form of sound, image or a combination of the above to indicate the user the condition of the environment. For example, the processing device 12 can inform the user the abnormal condition of the temperature by generating a speech or an image according to the retrieved actual environment status information 21.

In another embodiment, after retrieving the actual environment status information 21, the processing device 12 further performs an intelligent environmental control on at least one environmental control system (not illustrated) disposed in the environment where the processing device 12 is located according to the actual environment status information 21. The environmental control system mentioned above can be an air-conditioning system, an illumination system or a combination of the above.

In a usage scenario, the processing device 12 controls the environmental control system to adjust the environment to a comfortable condition when the abnormal condition, such as when the temperature is too high or when the brightness is too dark that cause physiological stimulation is detected according to the actual environment status information 21 and detects the breathing waveform of the user subsequently to guide the user to breath correctly in the comfortable environment.

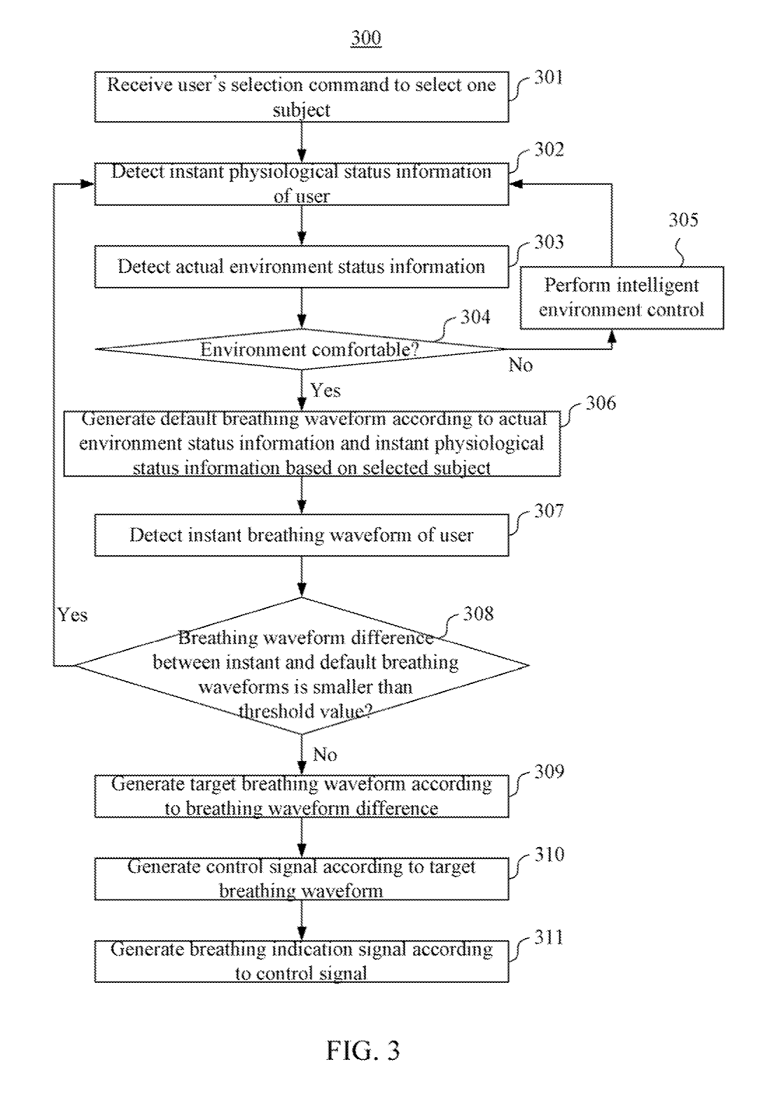

FIG. 3 is a flow chart of a breathing guidance method 300 in an embodiment of the present invention. The breathing guidance method 300 can be used in the breathing guidance system 2 as illustrated in FIG. 2. The breathing guidance method 300 includes the steps outlined below.

In step 301, the selection command is received by the processing device 12 to select one subject.

In step 302, the instant physiological status information 11 of the user is detected by the instant physiological status detection device 10. The number of the instant physiological status information 11 can be one or more than one.

In step 303, the actual environment status information 21 is detected by the environment sensor 20 of the processing device 12.

Whether the environment is comfortable is determined according to the actual environment status information 21 by the processing device 12.

When the environment is not comfortable, the intelligent environment control is performed by the processing device 12 in step 305. The flow then goes back to step 302 to keep performing detection.

When the environment is comfortable, the default breathing waveform is generated in step 306 according to the actual environment status information 21 and the instant physiological status information 11 based on the subject selected in step 301.

In step 307, the instant breathing status information 17 of the user is detected by the instant breathing detection module 16 and the instant breathing waveform is generated according to the instant breathing status information 17 and the default breathing waveform by the processing device 12.

In step 308, whether a breathing waveform difference between the instant breathing waveform and the default breathing waveform is smaller than a threshold value is determined.

When the breathing waveform difference is smaller than the threshold value, the flow goes back to step 302 to detect the instant physiological status information 11 again. When the breathing waveform difference is not smaller than the threshold value, the target breathing waveform is generated by the processing device 12 according to the breathing waveform difference in step 309 such that the control signal 13 is further generated in step 310.

In step 311, the breathing indication signal 15 is generated according to the control signal 13 by the breathing indication device 14.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A breathing guidance system having an active biofeedback mechanism, comprising:
a physiological status detection device to detect at least one of instant physiological status information of a user;
a processing device to generate a target breathing waveform according to the instant physiological status information and a default breathing waveform to further generate a control signal;
a breathing indication device to generate a breathing indication signal according to the control signal; and
an instant breathing detection module to detect instant breathing status information of the user;
wherein the processing device further generates an instant breathing waveform according to the instant breathing status information and the default breathing waveform, and the processing device compares the target breathing waveform and the instant breathing waveform to generate a breathing condition indication signal.

2. The breathing guidance system of claim 1 wherein the instant physiological status information comprises body movement information, oxygen saturation information, heartbeat rate information or a combination of the same.

3. The breathing guidance system of claim 2, wherein the physiological status detection device comprises a gravity sensor to detect body movement information, an oxygen saturation measuring device to detect oxygen saturation information, a heartbeat rate measuring device to detect heartbeat rate information or a combination of the same.

4. The breathing guidance system of claim 1, wherein the instant breathing detection module comprises a radar wave transceiver module, supersonic wave transceiver module or a laser transceiver module to transmit a radar wave, a supersonic wave or a laser to the user or receive the radar wave, the supersonic wave or the laser from the user to detect the instant breathing status information of the user.

5. The breathing guidance system of claim 1, wherein the instant breathing detection module comprises a sound-receiving module to receive a breathing sound of the user to detect the instant breathing status information of the user.

6. The breathing guidance system of claim 1, wherein the target breathing waveform and the instant breathing waveform each comprises a breathing depth, a breathing frequency or a combination of the above.

7. The breathing guidance system of claim 1, wherein the breathing indication device is a light generation device, and the breathing indication signal is a breathing indication light having an indication color temperature, an indication flashing frequency or a combination of the above.

8. The breathing guidance system of claim 1, wherein the breathing indication device is a sound generation device, and the breathing indication signal is a single frequency indication sound signal or a music indication sound signal that comprises an indication intensity, an indication frequency or a combination of the same.

9. The breathing guidance system of claim 1, wherein the processing device further comprises an environment sensor to sense actual environment status information, and the processing device generates the default breathing waveform according to actual environment status information and the instant physiological status information.

10. The breathing guidance system of claim 9, wherein actual environment status information comprises light intensity, temperature, humidity, carbon monoxide content, carbon dioxide content, volatile organic compound content, formaldehyde content or a combination of the above.

11. The breathing guidance system of claim 9, wherein the processing device further generates an indication message according to actual environment status information.

12. The breathing guidance system of claim 9, wherein the processing device further performs an intelligent environmental control on at least one environmental control system disposed in an environment where the processing device is located according to actual environment status information.

13. The breathing guidance system of claim 12, wherein the environmental control system is an air-conditioning system, an illumination system or a combination of the above.

14. The breathing guidance system of claim 1, wherein the processing device further determines that whether a breathing waveform difference between the instant breathing waveform and the default breathing waveform is smaller than a threshold value, and generates the target breathing waveform according to the breathing waveform difference when the breathing waveform difference is not smaller than the threshold value.

15. The breathing guidance system of claim 1, wherein the processing device further receives a selection command to select one of a plurality of default breathing waveforms.

16. A breathing guidance method used in a breathing guidance system having an active biofeedback mechanism, wherein the breathing guidance method comprises:
detecting at least one of instant physiological status information of a user by a physiological status detection device;
generating a target breathing waveform according to the instant physiological status information and a default breathing waveform by a processing device to further generate a control signal;
generating a breathing indication signal according to the control signal by a breathing indication device;
detecting instant breathing status information of the user by an instant breathing detection module; and
generating an instant breathing waveform according to the instant breathing status information and the default breathing waveform and comparing the target breathing waveform and the instant breathing waveform to generate a breathing condition indication signal by the processing device.

17. The breathing guidance method of claim 16, wherein the instant physiological status information comprises body movement information, oxygen saturation information, heartbeat rate information or a combination of the same.

18. The breathing guidance method of claim 16, wherein the step of detecting the instant breathing status information of the user by the instant breathing detection module further comprises:
transmitting a radar wave, a supersonic wave or a laser to the user or receiving the radar wave, the supersonic wave or the laser from the user by the instant breathing detection module to detect the instant breathing status information of the user.

19. The breathing guidance method of claim 16, wherein the step of detecting the instant breathing status information of the user by the instant breathing detection module further comprises:
receiving a breathing sound of the user by the instant breathing detection module to detect the instant breathing status information of the user.

20. The breathing guidance method of claim 16, wherein the target breathing waveform and the instant breathing waveform each comprises a breathing depth, a breathing frequency or a combination of the above.

21. The breathing guidance method of claim 16, wherein the step of generating the breathing indication signal by the breathing indication device further comprises:
generating a breathing indication light having an indication color temperature, an indication flashing frequency or a combination of the above by the breathing indication device.

22. The breathing guidance method of claim 16, wherein the step of generating the breathing indication signal by the breathing indication device further comprises:
generating a single frequency indication sound signal or a music indication sound signal that comprises an indication intensity, an indication frequency or a combination of the same by the breathing indication device.

23. The breathing guidance method of claim 16, further comprising:
sensing actual environment status information by an environment sensor comprised by the processing device;
generating the default breathing waveform according to actual environment status information and the instant physiological status information by the processing device.

24. The breathing guidance method of claim 23, wherein actual environment status information comprises light intensity, temperature, humidity, carbon monoxide content, carbon dioxide content, volatile organic compound content, formaldehyde content or a combination of the above.

25. The breathing guidance method of claim 23, further comprising: generating an indication message according to actual environment status information by the processing device.

26. The breathing guidance method of claim 23, further comprising: performing an intelligent environmental control on at least one environmental control system disposed in an environment where the processing device is located according to the actual environment status information by the processing device.

27. The breathing guidance method of claim 26, wherein the environmental control system is an air-conditioning system, an illumination system or a combination of the above.

28. The breathing guidance method of claim 16, further comprising:
determining by the processing device that whether a breathing waveform difference between the instant breathing waveform and the default breathing waveform is smaller than a threshold value; and
generating the target breathing waveform by the processing device according to the breathing waveform difference when the breathing waveform difference is not smaller than the threshold value.

29. The breathing guidance method of claim 16, further comprising receiving a selection command to select one of a plurality of default breathing waveforms by the processing device.
